(19) United States
(12) Patent Application Publication
(10) Pub. No.: US 2019/0333644 A1
(43) Pub. Date: Oct. 31, 2019

(54) METHOD AND SYSTEM FOR CREATING A CUSTOMIZABLE DENTAL CHART

(71) Applicant: CUMULUS DENTAL INCORPORATED, Ottawa (CA)

(72) Inventors: Marc Joseph Réne Lamarre, Ottawa (CA); Miguel Alberto Planas, Ottawa (CA); Sean Emberley, Nepean (CA)

(21) Appl. No.: 16/470,204

(22) PCT Filed: Dec. 14, 2017
(86) PCT No.: PCT/CA2017/051514

§ 371 Date: Jun. 15, 2019

Related U.S. Application Data
(60) Provisional application No. 62/435,124, filed on Dec. 16, 2016.

(51) Int. Cl. G16H 50/50; G16H 10/60

(52) U.S. Cl. CPC G16H 50/50; G16H 15/00; G16H 10/60

(57) ABSTRACT

The present invention provides a method and system for creating a customizable dental chart. The methods utilize independently modifiable representations of oral structures including teeth, soft and/or bony structures, wherein each of the representations of oral structures comprises a base structure and one or more attributes which optionally comprise one or more properties. The one or more attributes may be independently activated and the one or more properties of the attributes may be independently activated to form a representation of said patient's oral/dental cavity.

### FIELD

The present application relates to the field of dental charting and more particularly to an electronic system and method of dental charting.

### BACKGROUND

Dental practitioners maintain patient dental charts in order to monitor a patient's general oral health and oral health history over a period of time. Dental charts can include such information as tooth wear and positioning, information regarding prior dental work, soft tissue hard tissue and support tissue pathologies and specific treatment notes among any types of information that the dental practitioner may deem germane for the patient's general dental treatment. Many modern dental practices continue to maintain traditional, physical paper dental charts. These physical paper dental charts are typically stored in connection with a broader patient chart in traditional filing cabinets that can be compromised from a security perspective, damaged in a fire or flood and lost or misplaced. Dental charting is historically a time-consuming process, requires at least two people to perform: a dental practitioner performing the actual dental exam and orally reporting the results of that exam to an assistant who manually records the results of the dental exam in the form of a dental chart. Some estimates provide that completing a traditional dental chart, including both paper and digital formats, for a single patient can take approximately 20 to 35 minutes. Furthermore, it is widely accepted in the field that manual recording also leads to significant data recording errors and/or unreadable data. Finally, clear and concise communication of exam results and treatments with patients is critical, current charting system cannot be used for this purpose. There is thus a need in the art for a means to:
1. Reduce the time needed to perform a complete dental exam (hard and soft tissues) and record the results
2. Improve the method for recording the outcomes of the exams (charting)
3. Improve the accuracy and clarity of the information provided in the chart
4. Reduce the clinical resources required to perform a full charting exam
5. Create a charting interface that the patient will understand

The most difficult problem facing developers of digital dental charting systems is encoding the very large number of possible combinations of primary and secondary tooth characteristics possible for patient's teeth. For example, a given patient's tooth may be rotated, exhibit at least one or more caries (cavities) of various sizes and in a number of potential positions, exhibit one or more fillings each of which might be made of a different material and of different size and a number of potential locations on the tooth, exhibit a number of soft tissue characteristics such as bleeding, pus and gum recession all at the same time. Current art with respect to digital dental charting systems use simple iconography to display tooth characteristics in chart displays that emulate traditional paper charts. In addition, hard tissue and soft tissue data are currently captured in separate charts. As a result, current applications (a) are not capable of displaying the patient's teeth in a graphically realistic manner, since they are not able to address the very large number of graphic images that would be required to show all the combinatorial states, and (b) are not capable of providing views that summarize all of the primary and secondary characteristics of patient's teeth for both hard and soft tissues in a single display in an efficient and intuitive manner. In order to capture all of the information required for a given tooth, users of current applications must use a combination of (a) the simple unstructured icons for primary conditions—typical applications can only display one of these per tooth, (b) lists of other conditions using coded messages, and (c) free-form notes associated with a given tooth. An example of an application using this scheme is in FIG. 11. There are a number of problems associated with this methodology, including:
1. It is difficult for the practitioner to understand the total set of conditions associated with a given tooth because the information is scattered in different places on the screen, and/or in separate dialog boxes.
2. Because it is not clear which information is associated with which teeth, it is easy for the user to misread or misinterpret the information captured on the screen, thus increasing user errors and slowing the process of interpretation of the chart information.
3. The resulting charts are complex and abstract, and often use icons that have no equivalent verbal labels to the user. This makes the displays difficult to interpret, as well as increasing the difficulty in learning to use the product correctly.

### SUMMARY OF THE INVENTION

An object of the present invention is to provide a method and system for creating a customizable dental chart. In accordance with an aspect of the present invention there is provided, a method of recording dental information for a patient, said method comprising the steps of: (a) providing a model, wherein said model comprises independently modifiable representations of oral structures including teeth, soft and/or bony structures, wherein each of said oral structures comprises a base structure and one or more attributes which optionally comprise one or more properties, wherein the one or more attributes may be independently activated and the one or more properties of said attributes may be independently activated to form a representation of said patient's oral/dental cavity; (b) receiving input from a user, the input comprising one or more characteristic(s) of said patient's oral cavity; (c) independently modifying one or more of the modifiable representations of oral structures of said model based on said received user input and thereby generating a dental report for said patient, wherein each of said one or more base structures to be modified is modified by activating one or more of the one or more of the attributes and optionally activating one or more of the one or more of properties of any activated attributes to produce a representation of one or more oral structures of said patient; (d) optionally outputting the dental report, optionally on a visual display; and (e) optionally storing the dental report in a patient database file associated with the patient.

In accordance with another aspect of the invention, there is provided a method of updating a dental record comprising the steps of: (a) retrieving a dental report from a patient database file located in a first database, the patient database file associated with a patient, wherein the dental report includes a model comprises independently modifiable representations of oral structures including teeth, soft and/or bony structures, wherein each of said representations of oral structures comprises a base structure and one or more attributes which may be independently activated/deactivated, wherein each of the one or more attributes optionally comprises one or more properties which may be independently activated/deactivated; wherein one or more of said representations of oral structures optionally has previously been modified in light of one or more historical characteristic(s) of said patient's oral cavity; (b) receiving input from a user, the input comprising one or more characteristic(s) of said patient's oral cavity; (c) comparing said one or more characteristic(s) with said one or more historical characteristic(s) of said dental model with said input and if said one or more characteristic(s) differs from said one or more historical characteristic(s) independently modifying one or more of said representations of oral structures of said model based on said received user input by activating/deactivating one or more of the one or more of the attributes and/or activating/deactivating one or more of the one or more of properties of said one or more attributes to produce a current representation of one or more oral structures of said patient and thereby updating said dental report for said patient; (d) optionally outputting the dental report, optionally on a visual display; and (e) optionally storing the dental report in a patient database file associated with the patient.

In accordance with another aspect of the invention, there is provided a method of recording and/or displaying dental information for a patient, said method comprising the steps of: providing one or more modifiable representations of oral structures, wherein each of said one or more representations of oral structures comprises a base structure and one or more attributes which optionally comprise one or more properties, wherein the one or more attributes may be independently activated/deactivated and the one or more properties of said attributes may be independently activated/deactivated to form a representation of said patient's oral/dental cavity; and activating/deactivating one or more of the one or more of the attributes and optionally activating/deactivating one or more of the one or more of properties of any activated attributes to produce a representation of said dental information.

In accordance with another aspect of the invention, there is provided a method of displaying dental information for a patient, said method comprising the steps of: displaying a graphical presentation of one or more representations of oral structures, wherein each of said one or more representations of oral structures comprises a base structure and one or more attributes which optionally comprise one or more properties, wherein the one or more attributes may be independently activated/deactivated and the one or more properties of said attributes may be independently activated/deactivated to form a representation of said patient's oral/dental cavity.

### BRIEF DESCRIPTION OF THE FIGURES

The present invention will be better understood in connection with the following Figures, in which:

FIG. 1 is a diagram of the input and output data flow for at least one embodiment of a dental charting system in accordance with the present invention.

FIG. 2 is a diagram of the functional modules of at least one embodiment of a dental charting system in accordance with the present invention.

FIG. 3 is a diagram of the system architecture of at least one embodiment of a dental charting system and patient management system in accordance with the present invention.

FIG. 4 is another diagram of the system architecture of at least one embodiment of a dental charting system and patient management system in accordance with the present invention where the integrated software system makes use of Cloud services, via a managed web server module.

FIG. 5 is the first part of a patient workflow diagram for use in connection with at least one embodiment of a dental charting system in accordance with the present invention.

FIG. 6 is the second part of a patient workflow diagram for use in connection with at least one embodiment of a dental charting system in accordance with the present invention.

FIG. 7 is a diagram illustrating the functionality modules of one embodiment of an electronic dental chart and patient management system in accordance with the present invention.

FIG. 8 is a diagram of an information flow diagram of an electronic dental chart and patient management system in accordance with at least one embodiment of the present invention.

FIG. 9 provides exemplary symbols/icons of the dental condition (attribute) and a listing of properties (modifiers) associated with the attribute.

FIG. 10 provides an illustrative example of various views (3D and periodontal) displayed by the method of one embodiment the present invention.

FIG. 11 provides an illustrative example of a prior art electronic dental charting system.

### BRIEF DESCRIPTION OF TABLES

Table 1—Non-limiting examples of 3D dentition features of the system of an embodiment of the invention.

Table 2—Non-limiting examples of conditions that may have anatomically correct display in the model of an embodiment of the present invention.

Table 3—Non-limiting examples of conditions may be without visual features to be displayed in the model in one embodiment of the invention

Table 4—Non-limiting examples of alternative presentation of the clinical information on the model in one embodiment of the invention.

Table 5—Non-limiting examples of clinically relevant information other than conditions

Table 6—Non-limiting examples of common dental exam device outputs.

Table 7—Non-limiting examples of clinically relevant information may be read from 3^{rd} party dental systems including but not limited to DPMS/DOMS.

Table 8—Non-limiting examples of dental pathologies and anomalies.

Table 9—Non-limiting examples of dental restorations.

### DETAILED DESCRIPTION

The present invention relates to electronic dental recording systems and methods, including systems and methods for dental charting. The electronic methods and systems of the present invention allows users to record, review, modify and store the information necessary for the management of the dental and oral health of its dental patients. The systems and methods of the present invention may simplify at least some of the aspects of clinical information management in the dental practice, including performing and recording complete dental exams, recording relevant clinical information past and present, visualizing clinically relevant information for reviews/treatment planning/patient education, and storing/retrieving/safeguarding clinical data. In addition, the systems and methods of the present invention may reduce the resources required to perform and record a dental exam, may improve the visualization and thus the interpretation of clinical information by the practitioner, and/or provide Canadian and United States of America compliant (current and future) clinical information management and security.

The systems and methods of the present invention separates various dental/oral characteristics (including but not limited to physiological features, pathological conditions and restorations/treatments) into discrete attributes and utilizes these attributes in combination with one more base structures/icons and optionally attribute properties (such as size, location, etc of the attribute) to produce composite representations of dental/oral characteristics.

In certain embodiments, the attributes are in the form of a graphic/icon. In certain embodiments, the attributes are in the form of overlay graphics or icons which may be displayed on a base icon. In other embodiments, the attributes are in the form of graphics or icons which replace the base icon. In other embodiments, the attributes are in the form of text. In certain embodiments, the attributes are in graphic and text forms.

These attributes optionally comprise one or more properties that further define the attribute. In certain embodiments, the properties are in the form of a graphic/icon. In other embodiments, the properties are in the form of text associated with the attributes.

Accordingly, the systems and methods of the present invention allow for the construction of composite(s) that contain all of the characteristics of a given oral cavity structure (such as a given tooth) as independently controllable elements following a systematic dictionary and grammatical structure (a language). This eliminates the need for defining and storing separate representations for each combinatorial state of that oral structure. The prior art does not apply this separation of information contents. Accordingly, the prior art methods need to provide separate representations for each combinatorial state. This results in millions of representations that would be virtually impossible to produce and manage digitally.

The system and methods of the present invention allows for the independent application of representations that provide both three-dimensional images (optionally graphically realistic 3D images) of the patient's oral structures (including but not limited to dentition), as well as a summary of all conditions for all of the patient's oral structures (for example in the form of a summary table or chart).

In addition, the system and method of the present invention simplifies the commands needed for the operator to specify the characteristics of each oral structure. This may facilitate the input of data manually and may facilitate the use of voice-recognition technology.

In certain embodiments, there is provided a method of displaying dental information for a patient, the method comprising the steps of: displaying a presentation of one or more representations of oral structure(s), wherein each of said one or more representations of oral structures comprises a base (generic or patient specific) structure and one or more attributes which optionally comprise one or more properties, wherein the one or more attributes may be independently activated/deactivated and the one or more properties of said attributes may be independently activated/deactivated to form a representation of the patient's oral/dental cavity. In specific aspects, the base structure comprises a base graphical representation, and each of said attributes comprises a graphical overlay.

In certain embodiments, there is provided a method of recording and/or displaying dental information for a patient, the method comprising the steps of: providing one or more modifiable representations of oral structures, wherein each of said one or more oral structures comprises a base (generic or patient specific) structure and one or more attributes which optionally comprise one or more properties, wherein the one or more attributes may be independently activated/deactivated and the one or more properties of said attributes may be independently activated/deactivated to form a representation of the patient's oral/dental cavity; and activating/deactivating one or more of the one or more of the attributes and optionally activating/deactivating one or more of the one or more of properties of any activated attributes to produce a representation of said dental information.

In certain embodiments, there is provided a method of recording dental information for a patient, said method comprising the steps of: (a) providing a model, wherein said model comprises independently modifiable representations of oral structures including teeth, soft and/or bony structures, wherein each of said representations of oral structures comprises a base (generic or patient specific) structure and one or more attributes which optionally comprise one or more properties, wherein the one or more attributes may be independently activated and the one or more properties of said attributes may be independently activated to form a representation of said patient's oral/dental cavity; (b) receiving input from a user, the input comprising one or more characteristic(s) of said patient's oral cavity; (c) independently modifying one or more of the representations of oral structures of the model based on said received user input and thereby generating a dental report for said patient, wherein said each of said one or more base structures to be modified is modified by activating one or more of the one or more of the attributes and optionally activating one or more of the one or more of properties of any activated attributes to produce a representation of said one or more oral structures of said patient; (d) optionally outputting the dental report, optionally on a visual display; and (e) optionally storing the dental report in a patient database file associated with the patient.

Also provided is a method of updating a dental record comprising the steps of: (a) retrieving a dental report from a patient database file located in a first database, the patient database file associated with a patient, wherein the dental report includes a model comprising independently modifiable representations of oral structures including teeth, soft and/or bony structures, wherein each of the representations of oral structures comprises a base (generic or patient specific) structure and one or more attributes which may be independently activated/deactivated, wherein each of the one or more attributes optionally comprises one or more properties which may be independently activated/deactivated; wherein one or more of said oral structures optionally has previously been modified in light of one or more historical characteristic(s) of said patient's oral cavity; (b) receiving input from a user, the input comprising one or more characteristic(s) of said patient's oral cavity; (c) comparing said one or more characteristic(s) with said one or more historical characteristic(s) of said dental model with said input and if said one or more characteristic(s) differs from said one or more historical characteristic(s) independently modifying one or more of the representations of oral structures of the model based on said received user input by activating/deactivating one or more of the one or more of the attributes and/or activating/deactivating one or more of the one or more of properties of said one or more attributes to produce a current representation of said one or more oral structures of said patient and thereby updating said dental report for said patient; (d) optionally outputting the dental report, optionally on a visual display; and (e) optionally storing the dental report in a patient database file associated with the patient.

Also provided is a computer program product comprising a computer readable memory storing computer executable instructions thereon that when executed by a computer perform the method of the invention.

Also provided is a system for electronic charting dental information for a patient, said system comprising a processor and a computer readable storage media comprising executable instructions thereon that when executed by said processor perform the method of the invention

### Dentition Model

The models of the present invention comprise independently modifiable representations of oral structures including teeth (wherein each tooth is independently modifiable), soft structures (including but not limited to nerves, major vessels and oral cavity anatomy) and/or bony structures. Specifically, in certain embodiments, the independently modifiable representations of oral structures comprise a base structure and one or more attributes which optionally comprise one or more properties. The attributes and attribute properties may be activated/deactivated to form a composite representation of a patient's oral/dental cavity.

It is contemplated that the model can be a model of any one of the following: Teeth Lower Jaw Adult, Teeth Upper Jaw Adult, Teeth Lower Jaw Children, Teeth Upper Jaw Children, Teeth Upper Jaw Mixed Dentition, Teeth Lower Jaw Mixed Dentition, Complete Upper and Lower Jaw Adult, Complete Upper and Lower Jaw Children, or Complete Upper and Lower Jaw Mixed Dentition. The models may be a model of standard dentition or a custom model, such as pathological model, or animal dentition models (such as canine and feline models) for use in veterinary practice.

Accordingly, in certain embodiments, the base structure may be generic. In other embodiments, the base structure is patient specific. For example, the base structure may be constructed from 2D or 3D scans of the patient's oral cavity.

In certain embodiments, the model is a standard model of adult dentition. In other embodiments, the model is a standard model of child dentition. In certain embodiments, the model is age appropriate and can be changed as the patient matures.

The models may include representations of dentition, soft tissue (including gum levels, nerve and/or blood vessels) and/or bone levels. In certain embodiments, the models may be manipulated such that underlying features which are normally hidden can be seen. The models may be an anatomically correct representation, two dimensional (2D), three dimensional (3D), line drawings, X-ray rendered view or a pictographic chart. The point of view of the model may be changed and/or the view of the model may be zoomed in or out and/or structures may be viewed individually. In certain embodiments, the model is a 3D anatomically correct representation. In other embodiments, the model is a pictographic chart. In such, embodiments, each tooth is represented individually and includes dental symbols/icons and optionally text. In specific embodiments, the model is a pictographic chart, wherein each tooth is represented individually and tooth attributes are one or more iconic representations and attribute properties are one or more textual representations.

In certain embodiments, the system and method allows for multiple types of models to be displayed simultaneously; for example, a 3D representation and a pictograph model may be displayed simultaneously.

In certain embodiments, the system and methods offer an X-Ray rendering of all the hard tissues and restorations found on the 3D chart with accurate radiolucency levels for all materials.

In certain embodiments, the system and methods have the ability to convert the 3D chart to a 2D charting rendering.

In certain embodiments, the system and methods have the ability to convert the 3D chart to a line drawing rendering.

In certain embodiments, the system and methods have the ability to convert the 3D chart to a natural looking rendering to mimic what is actually seen in the mouth.

In certain embodiments, the system and methods have the ability to strip away all of the soft tissues and display the bone levels around the dentition.

In certain embodiments, the system and methods have the ability to strip away all of the bone and soft tissues and display only the dentition.

In certain embodiments, the system and methods display an animation of the treatment history of the patient's mouth.

In certain embodiments, the system and methods display a periodontal view to record the results of an exam on the soft tissues.

In certain embodiments, the models display one or more of the following features Tooth Crown Anatomy, Tooth Root Anatomy, Cemento-enamel Junction (CEJ), Dental Pulp Chamber, Root Pulpal Canals, Gingival Contours and Alveolar Bone Levels.

### Attributes and Properties of Attributes

An attribute may be any characteristic of a patient's oral cavity that the practitioner can observe when performing a traditional dental exam. This can include, without limitation, any information related to any aspect of a patient's oral health that the dental practitioner may be able to observe, obtain or measure and can relate to, for example, information relating to health and characteristics of hard, soft and support tissue as discussed in further detail below.

In certain embodiments, attributes include are not limited to one or more of the following dental pathologies and anomalies: Dental Caries, Recurrent Decay, Periapical Abscesses, Gingival or Periodontal Abscesses, Gingival Ulcers, Broken teeth, Retained roots, Impacted Teeth, Supernumerary Teeth, Submerging Teeth, Retained Deciduous Teeth, Gingival Recessions, Gingival Bleeding, Gingival Pus, Furcation Involvements, Gingival Redness, Gingival Hypertrophy, Gingival Hyperplasia, Rotated teeth, Tipped Teeth, Overerupted Teeth, Fistulas, Tooth Abrasion, Tooth erosion, Occlusal wear, Odontomas, Tooth fusions, Tooth germinations and Diastemas.

In certain embodiments, attributes include but are not limited to one or more of the following dental restorations: Amalgams, Composite Resins, Porcelain Fused to Metal crowns, Full Gold Crowns, Full Metal Crowns, Porcelain Jacket Crowns, Full Porcelain Crowns, Porcelain Veneers, ¾ Metal Crowns, Metal Inlays, Porcelain Inlays, Bridge Abutments, Bridge Pontics, Dental Implants, Maryland Bridges, Endodontic Treatments, Endodontic posts and Pit and Fissure Sealants.

In certain embodiments, attributes include the absence of a feature, such as absence of a tooth. In certain embodiments, the one or more attributes relate to the other aspects of oral health that are related to, but distinct from, the patient's tooth itself (such as, for example, a proximally located tumour or local presence of gingivitis).

Properties of the attributes further define the attribute and include but are not limited to size, location, degree of rotation, material, colour and texture.

For example, in order to record a large dental caries involving the occlusal surface of top right first molar and a composite resin filling of the occlusal surface of the top left second molar on the system of the invention, a user may: (1) activate the attribute “dental caries” for the representation of the top right first molar, activate the following properties of this dental caries: size incipient or in dentin and location occlusal surface. (2) activate the attribute “filling” for the representation of the top left second molar, activate the following properties of this filling: material composite resin and location occlusal surface. Following treatment of the large dental caries with a composite resin filing, the user may make the following updates: as this would be a new session, he would simply now activate the attribute “filling” to the top right first molar, activate the following properties of this filling: material composite resin.

The systems and methods of the present invention also allow for recordation of a patient's oral cavity/dentition characteristics for which there is no visual display. Non-limiting examples include pocket depth, temperature, tooth mobility and halitosis. In certain embodiments, such non-graphical condition/medical information may be presented on the charting page using context appropriate digital means including but not limited to bubbles, menus, floating text box, without replacing the charting page.

### User Input

It is contemplated that input from the user can take a wide variety of formats and can be inputted into the present method and system by any suitable input means. Accordingly, it will be readily appreciated that input may be from a single source or multiple sources.

Examples of types of suitable input format can include, but are not limited to, voice input, keyboard input, mouse input, touchscreen input and electronic input from a suitable device, among any other types of input that will be readily understood by the skilled person.

In certain embodiments, at least part of the input is voice input, optionally by way of a wired or wireless microphone. A worker skilled in the art would readily appreciate that voice input would be processed by a voice recognition module.

In certain embodiments, at least part of the input is keyboard or touchscreen input. In certain embodiments, at least part of the input is electronic input.

In certain embodiments, at least part of the input is input from external devices or systems including but not limited input from a 3^{rd} party dental systems (such as dental practice/office management systems)); input from cloud services; and/or input external exam modalities including but not limited to dental probing systems, imagining systems including but not limited to cameras, MRI, CAT, ultrasound and X-ray machines.

In certain embodiments, at least part of the input is from intraoral Cameras, Digital periapical radiographs, Digital panoramic radiographs, Digital lateral cephalometric radiographs, Optical Coherence Tomography (OCT), CAT scans, Probes.

### Modifying One or More Representations of Oral Structures of the Model Based on User Input

One or more representations of oral structures may be modified based on the received user input by activating/deactivating one or more of attributes and one or more attribute properties to produce a composite representation of one or more oral structures of said patient. A worker skilled in the art would readily appreciate that a single oral structure, such as a tooth, may have more than one characteristic. For example, a worker skilled in the art would readily appreciate that a single tooth may include multiple dental restorations or dental caries or a combination of a dental restoration and a dental caries. Accordingly, the system and methods of the present invention allows for the application of more than one attribute to a single representation of an oral structure. Likewise, a worker skilled in the art would readily appreciate that certain dental restorations involve multiple structures (such as a bridge). Accordingly, the systems and methods of the present invention allow for a single attribute to be applied to multiple representations of oral structures.

In addition, the systems and methods of the present invention also allow for recordation of non-structural characteristics of the patient's oral cavity or dentition. Non-limiting examples include pocket depth, temperature, tooth mobility and halitosis.

In methods of updating the dental chart, previously activated attributes or the properties of these attributes may be deactivated in light of changes to the patient's oral cavity/dentition. In addition, further attributes/properties may be activated in light of changes to the patient's oral cavity/dentition.

### Outputting and Saving Dental Chart

The methods and systems of the present invention optionally produce an output report which may be in the form of a dental chart and optionally other non-clinical information. The report may be displayed in the host visual display. For example, the dental report may be embedded within a software product that is accessed by any number of devices having a visual display and input means, including but not limited to a mobile device, desktop computer, tablet computer and wearable device among any other device that will be readily understood by the skilled person. Alternatively, the report may be in a printed (i.e. paper) or printable format (such as a PDF format). Also contemplated is a 3D print out of the modified model of the patient.

The dental chart report be presented in a variety of ways depending on the needs of the end user application. For example, it is contemplated that the dental report can be in the form of a traditional dental chart (i.e. two-dimensional format), a three-dimensional format more akin to the actual presentation of the patient's oral features or as a pictograph chart. The report may include a representation of all or a portion of the oral structures and may also include non-structural characteristics.

Any suitable digital storage medium may be used. Such digital storage mediums include but are not limited to remote storage (such as a remotely accessible cloud-based server), removable storage, local storage (such as local storage on a tablet or computer device) and secure storage among any other arrangement that will be readily understood by the skilled person.

It is contemplated that all storage and communication of any digital information in connection with the present invention will be governed by and in accordance with any applicable health and privacy laws that may apply in the instant jurisdiction, including but not limited to The Canadian Personal Protection and Electronic Documents Act (“PIPEDA”) and/or the Health Insurance Portability and Accountability Act (“HIPAA”) of America.

In at least one embodiment it is contemplated that the electronic dental chart is stored in a corresponding patient file that is associated with a particular patient and stored in the database. It is contemplated that the patient file is accessed by the dental practitioner through an electronic dental charting and patient management system that can further include additional cooperating and interconnected functionality modules such as but not limited to a patient schedule module, a patient treatment module for diarizing past and future treatment, a patient payment module for tracking outstanding and paid invoices, a patient charting module, an input translator module, a command/task sorting module, a display manager module, a data storage module, exam notes recordation module, dentition exam selection and dentition display module, a 3^{rd} party data display module, an alternate display generator module, a recorder module for a dental examination tool such as a dental examination probe, a Dental Practice/Office Management System clinical information module, and a Dental Practice/Office Management System call module, a voice recognition module, a data extraction module, a data matching module and a dental chart generation module and a communication module for providing a communication link to a third party service provider such as a health insurer or the patient's email service among any other suitable functionality modules that will be readily appreciated by the skilled person. It is further contemplated that the electronic dental chart can interface with these additional and optional functionality modules.

In this way, the present invention provides a full patient management tool that is capable of tracking each aspect of a patient's dental treatment in a single software program in order to provide more efficient and personalized dental health care.

It is contemplated that all storage and communication of any digital information in connection with the present invention will be governed by and in accordance with any applicable health, privacy and regulatory laws that may apply in the instant jurisdiction, including but not limited to The Canadian Personal Protection and Electronic Documents Act (“PIPEDA”) and/or the Health Insurance Portability and Accountability Act (“HIPAA”) of America.

It is also contemplated that the present invention will comply with any International Standards Organization, Food and Drug Administration, Health Canada or other applicable standards for software intended for use in medical/dental applications.

### Additional Information

In certain embodiments, the methods and systems of the present invention allow for input of additional information. This information may be clinically relevant information or non-clinically relevant information. Non-limiting examples of clinically relevant information includes but is not limited to medical history, dental history, personal medical information, medical history updates and dental treatment plan. Non-limiting examples of non-clinically relevant information includes but is not limited to patient photo, patient contact information, family information, financial information, insurance information and scheduling. In certain embodiments, this additional information is obtained from a 3^{rd} party dental systems

### External Interfaces:

The systems of the present invention may be a stand-alone system, interfaced with one or more other systems or integrated in another system.

In certain embodiments, the systems and methods may interface with other systems and methods. Such other systems include but are not limited to a 3^{rd} party dental systems (such as dental practice management systems); cloud services; and/or external exam modalities including but not limited to dental probing systems, imagining systems including but not limited to cameras, MRI, CAT, ultrasound and X-ray machines.

In certain embodiments, the systems and methods of the present invention interface with a 3^{rd} party dental management systems. Such an interface may allow access to clinical and non-clinical information (scheduling, billing, practice financials, resources management, etc.) thereby streamlining operations.

In certain embodiments, the systems and methods of the present invention interface with cloud services. Such an interface would allow for remote data storage and/or remote access a patient's records thereby allowing multiple off-site locations to share patient records.

In certain embodiments, the systems and methods of the present invention interface with one or more external exam modalities including but not limited to dental periodontal and physiological probing systems (Probes), imagining systems including but not limited to cameras, MRI, CAT, ultrasound and X-ray machines.

In certain embodiments, the systems and methods of the present invention interface with one or more of Intraoral Cameras, Digital periapical radiographs, Digital panoramic radiographs, Digital lateral cephalometric radiographs, OCT—Optical Coherence Tomography, CAT scans and Probes.

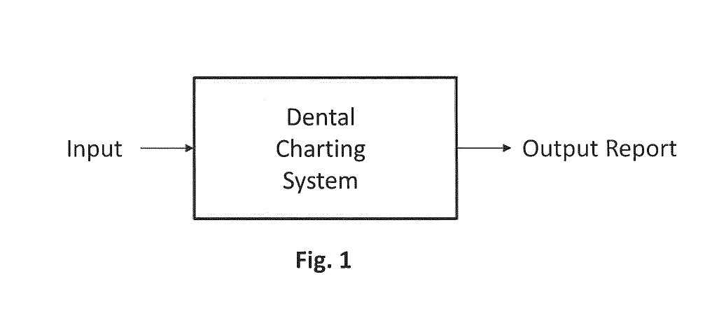

Turning to FIG. 1, at least one embodiment of the present invention is illustrated wherein the present dental charting system receives input from the practitioner and outputs a completed patient dental chart.

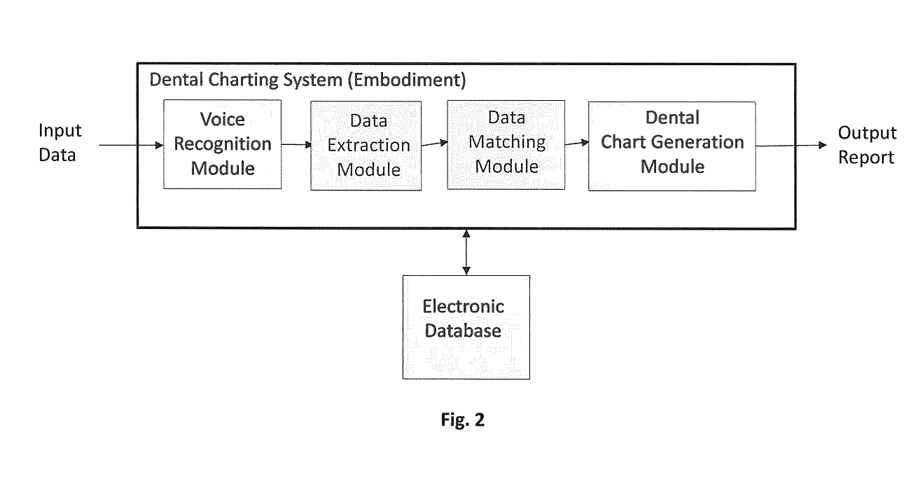

Turning to FIG. 2, at least one embodiment of the present invention is illustrated wherein the present dental charting system includes various co-operating functionality modules including a voice recognition module for deciphering input data from the user, a data extraction module for extracting user data from the deciphered input data, a data matching module for matching the extracted data to a previously populated database and a dental chart generation module for displaying a resultant electronic dental chart. It is further contemplated that the dental charting system can communicate with a database, and further that each module can communicate with one another in a parallel or series manner, as shown.

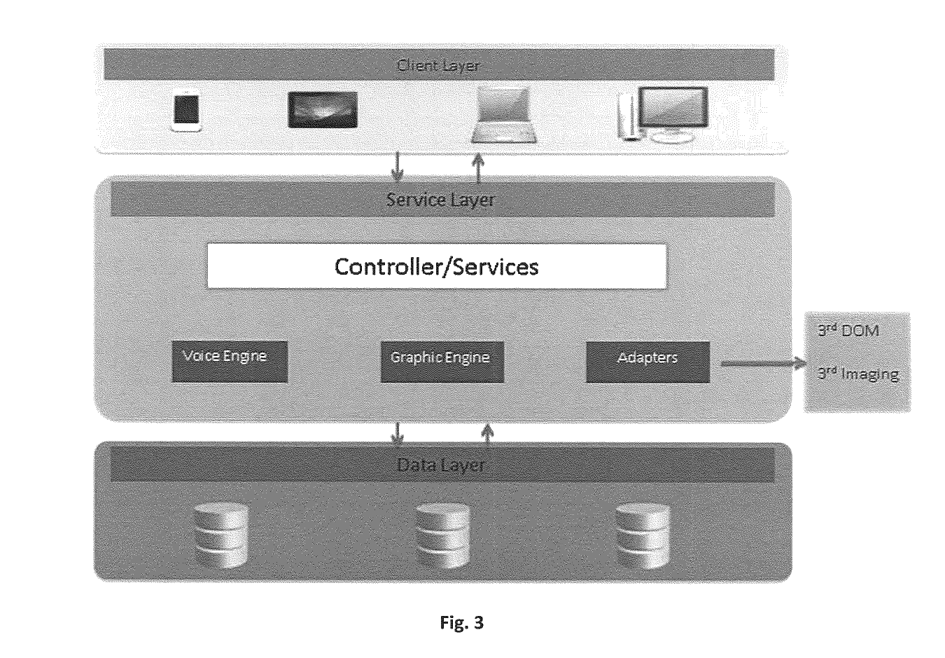

Turning to FIG. 3, a diagram of the system architecture of at least one embodiment of the present invention is illustrated wherein a client layer (which can include any user device having input means for inputting data into the present electronic dental charting and patient management system) communicating with a remotely located service layer at a first location (and containing data processing modules such as, but not limited to, a graphics engine for displaying graphics, voice recognition engine for deciphering inputted vocal data, a third party Dental System (“such as a Dental Practice Management System or DPMS) adapter and a third party imaging adapter for displaying images, among other data processing modules that will be readily understood by the skilled person). In turn, the service layer can further communicate with a data layer that provides remote storage. It is contemplated that the data layer can be located at a second location or co-located with the service layer, among other arrangement that will be readily understood by the skilled person).

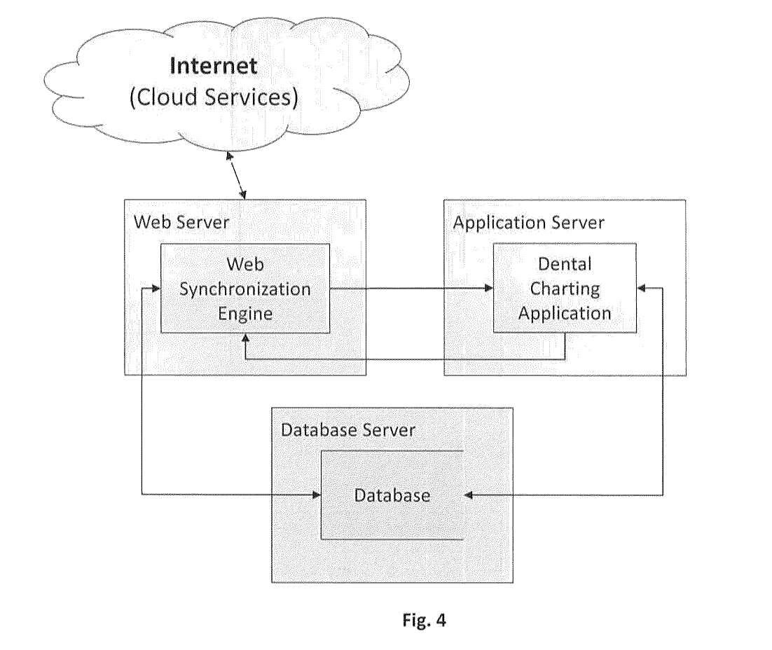

Turning to FIG. 4, another diagram of the system architecture of at least one embodiment of the present invention is illustrated wherein a web browser communicates with a web application running on a web server through a communications network such as the internet, a wide area network or a local area network.

An application server can be provided that hosts the dental charting application and which can receive input data from the web application and send output data to the web application. It is further contemplated that both the web application and the charting application can communicate with a remotely located database hosted on a database server.

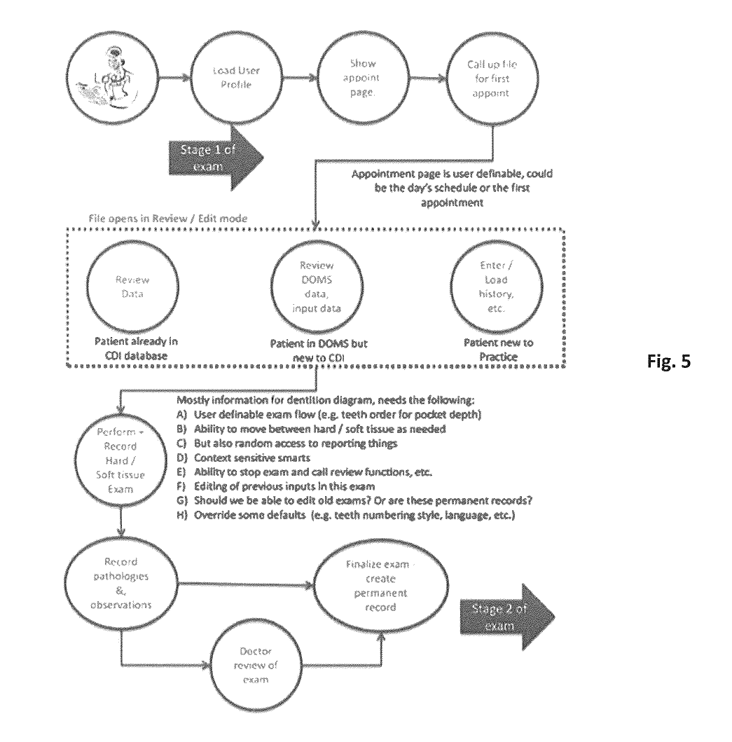

Turning to FIG. 5 at least one embodiment of a sample work flow during a patient exam is illustrated in accordance with at least one embodiment of the present invention. A user logs (the login optionally is password protected) into the electronic patient suite and loads a user profile for that particular practitioner. The user can access a patient file in the database. The patient file includes a model which comprises independently modifiable representations of oral structures including teeth, soft and/or bony structures (which for existing patients may include previously modified oral structures. For new patients, a new patient file that needs to be created and completed with data that is inputted by the user or alternatively an imported patient file that is imported into the present dental charting system from another dental charting system.

Creation of a new patient file includes selection of an appropriate model which comprises independently modifiable representations of oral structures. Next, the user performs the dental exam. During the exam, the user inputs data comprising at least one characteristic of one or more structures in the patient's oral cavity and one or more oral structures of the model are modified based on the received user input. As will be understood by the skilled person, any inputted characteristic can be inputted into the present electronic dental charting system as discussed in detail above. For example, at any point during the exam the user can input any pathologies, any dental restorations, observations or other information that the user has related to a particular patient's exam. Further, it is contemplated that the user can examine the patient's teeth in any order (such as, in sequence, in a pre-established protocol or in a completely random manner). The exam can be stopped at any time to review and edit any inputted information and override any defaults that are preloaded into the system.

Once the electronic dental chart has been modified based on the inputted characteristics, the user may review the results of the exam and modify the electronic chart further. Once the electronic dental chart has been reviewed, the exam may be finalized and stored in the patient file located in an electronic database that may be located either remotely or locally.

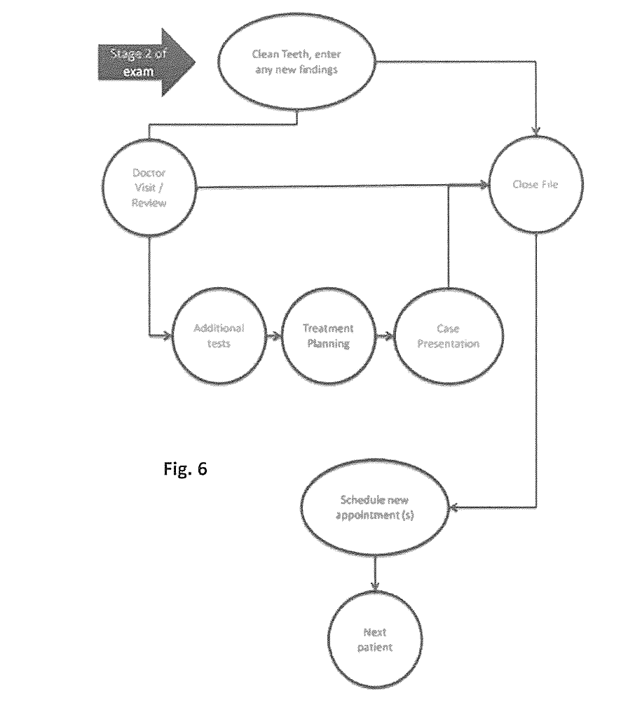

Turning to FIG. 6, at least one embodiment of a second aspect of the sample work flow during a patient exam is illustrated in accordance with at least one embodiment of the present invention.

In this embodiment, the user completes any treatment that is required for that particular patient (such as, for example, a dental cleaning), the user can modify the electronic dental chart and the patient file to include any new information related to treatment planning, any additional tests and case presentations, among any other information that is pertinent to the particular patient. These modifications can be saved and the patient file can be closed.

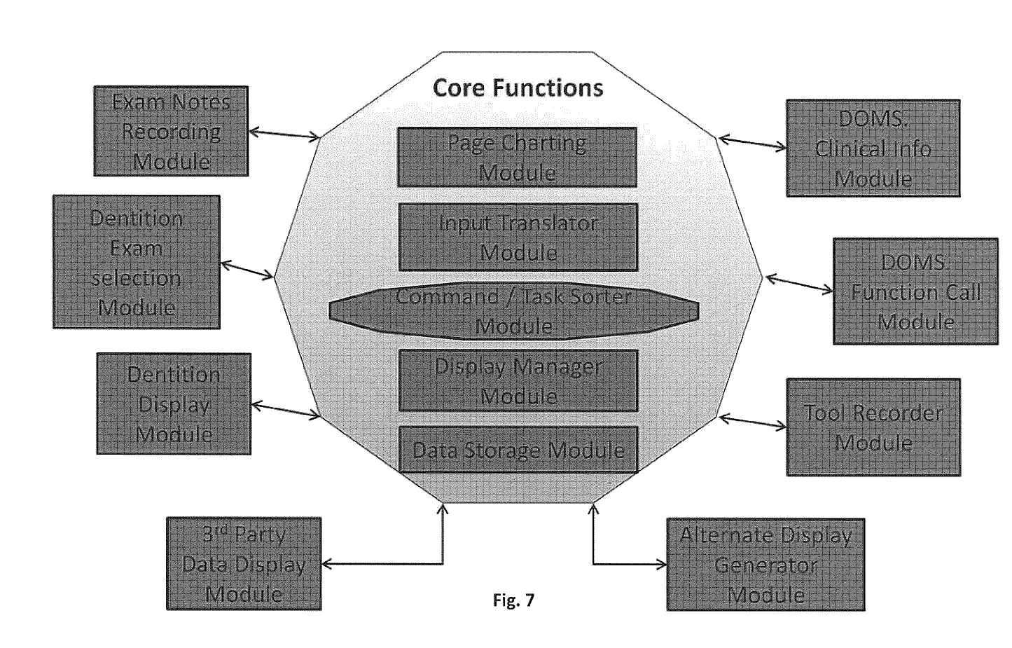

Turning to FIG. 7, functionality modules of one embodiment of the electronic dental chart is illustrated. Core functional modules of the electronic dental chart can include the electronic dental chart display module for displaying the electronic dental chart, an input translation module for deciphering input data, a command/task sorter module, a display manager module, and a data storage module, among any other types of functional modules that will be readily understood by the skilled person. Additional functional modules of the electronic dental charting and patient management system can include an examination notes recordation module, dentition exam selection and dentition display modules, a 3^{rd} party data display module, an alternate display generator module, a recorder module for a dental examination tool such as a dental examination probe, a Documentation Object Module clinical information module, and a Documentation Object Module function call module, among any other types of functional modules that will be readily understood by the skilled person.

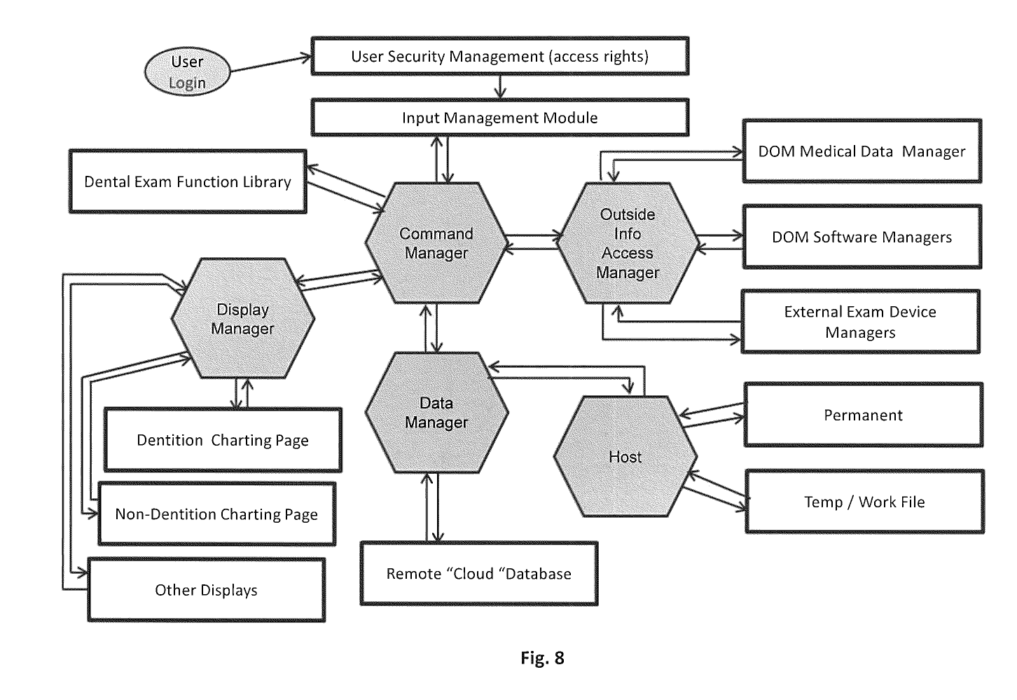

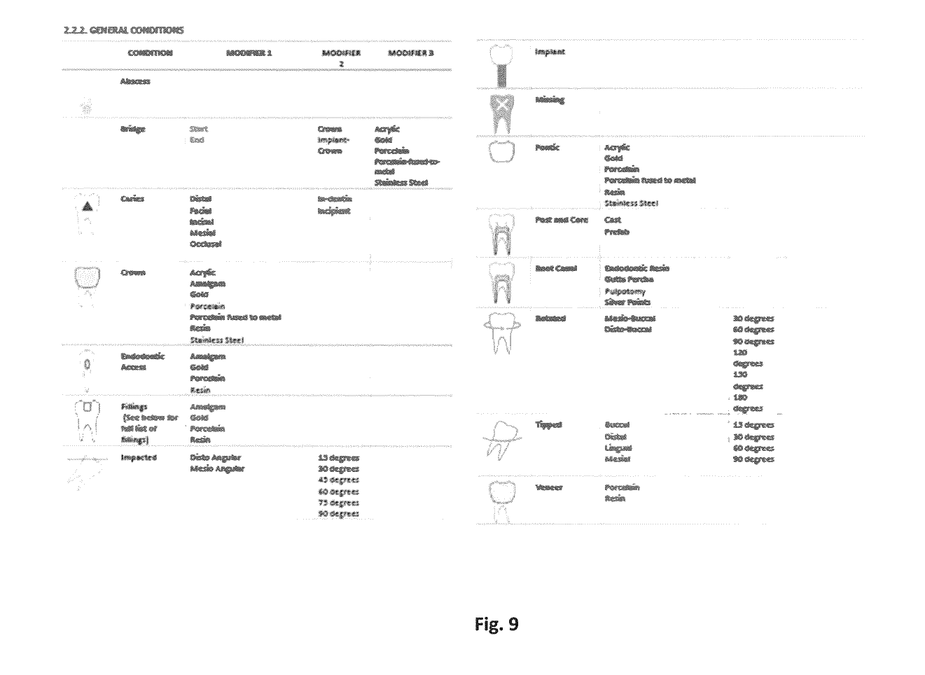

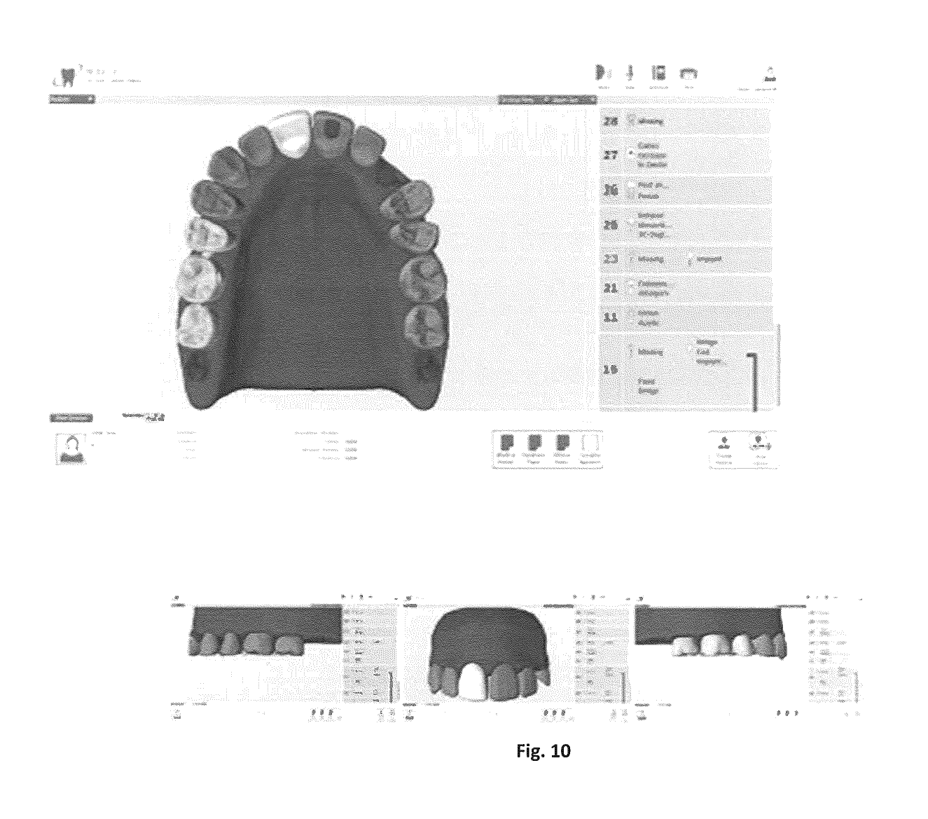

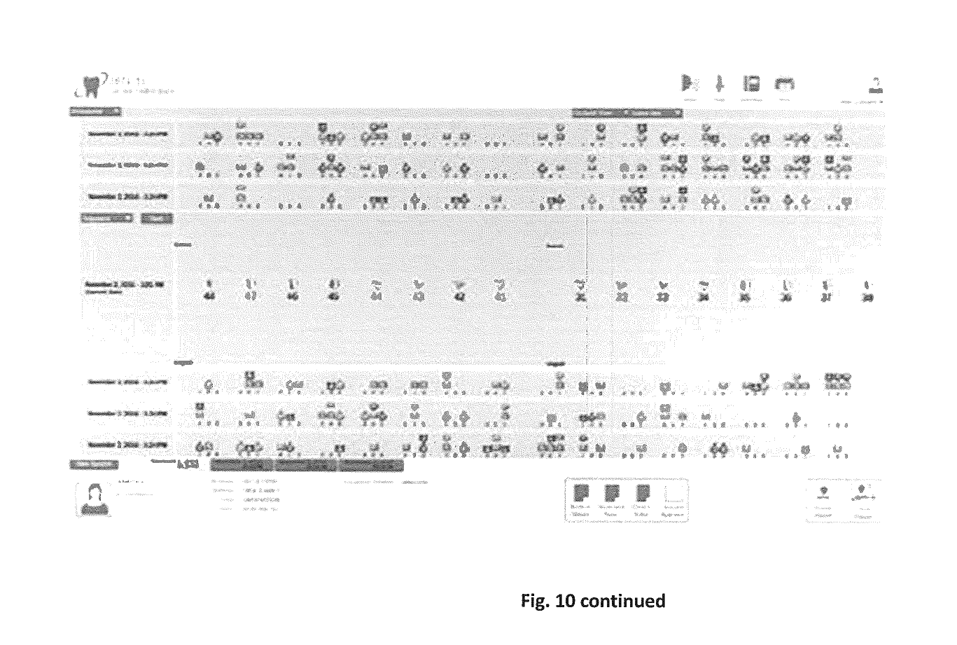

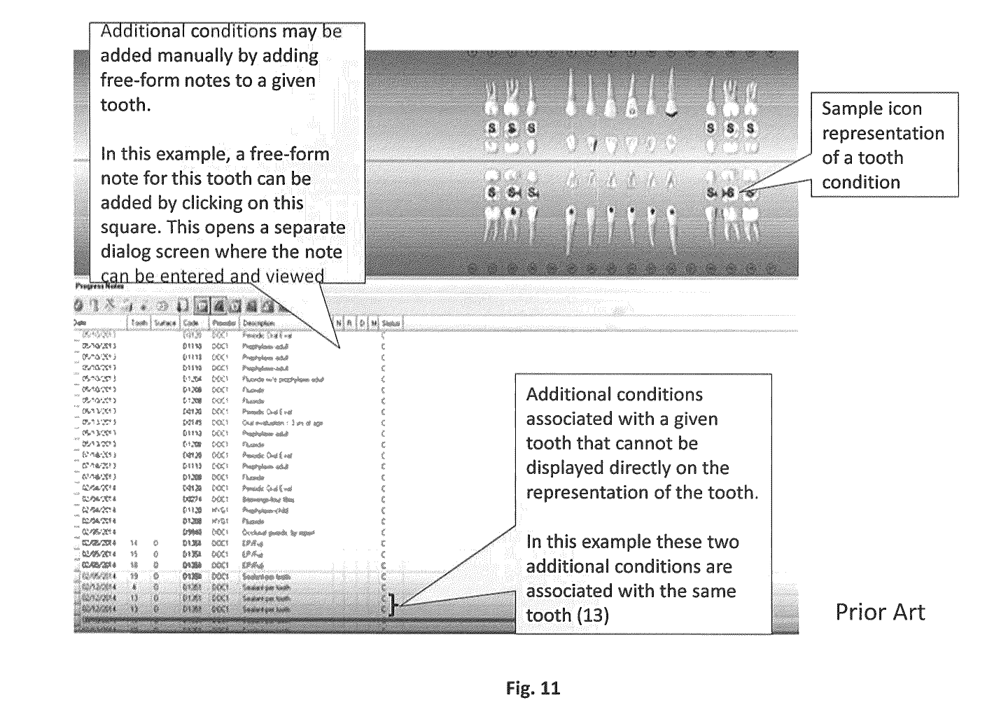

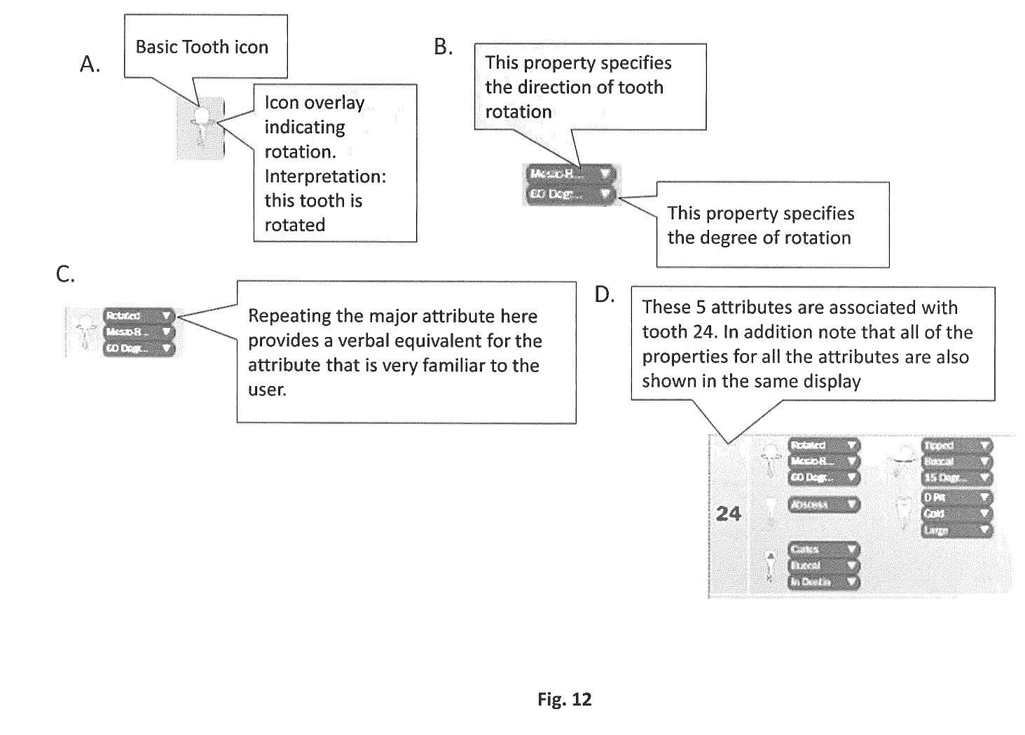

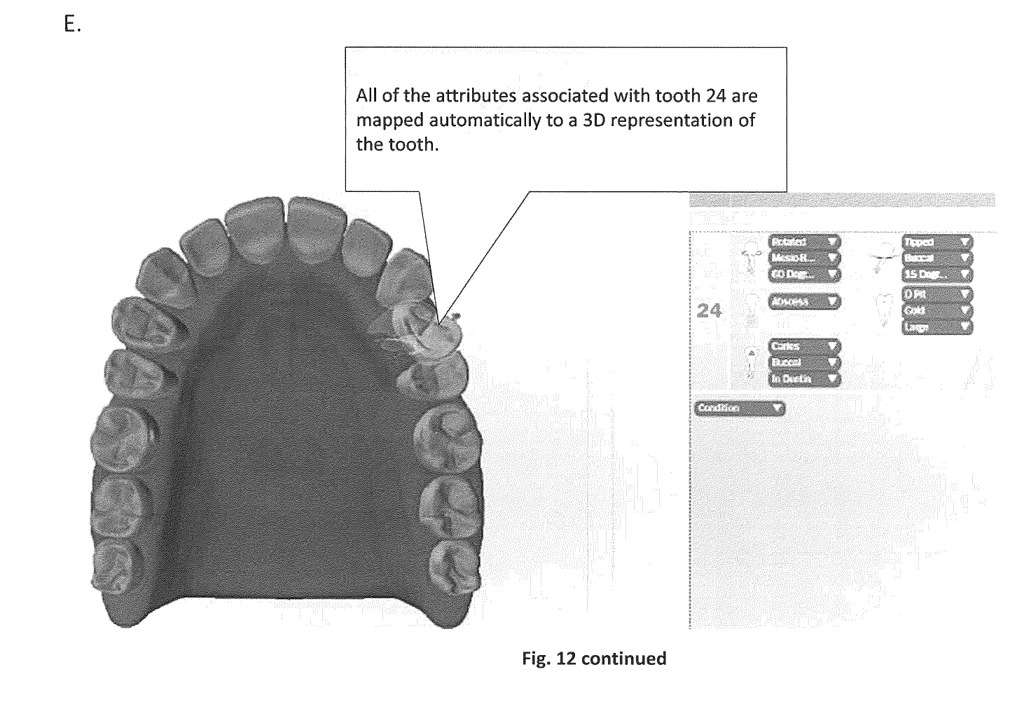

Turning to FIG. 8, one embodiment of an information flow diagram in accordance with at least embodiment of the present invention is illustrated. In this embodiment, the user login is accomplished through a user security management module and an input management module that communicates with a command manager. The command manager in turn communicates with, for example, a dental exam function library, a display manager (which can in turn communicate with, for example, a dentition charting page, a non-dentition charting page and any other display that will be readily understood by the skilled person) a data manager (which in turn can communicate with, for example, a remote cloud database and a host communicating with, for example, a temporary or a permanent work file) and an outside information access manager (which can communicate with, for example, a Document Object Modeling data manager, a Document Object Modeling software manager and any suitable external examination device managers).

Turning to FIG. 12, in an exemplarily embodiment an icon representing tooth 24 is provided with an overlay graphic identifying that the tooth is rotated (FIG. 12A). Text representation indicates the direction of rotation and degree rotation (FIG. 12B). The iconic and textual representations can be combined to provide a compact and understandable description of one characteristic of tooth 24 (FIG. 12C). Using this approach a large number of characteristics associated with a given oral structure can be displayed in a compact manner. For example FIG. 12D illustrates tooth 24 is rotated 60 degrees mesio-buccal, tipped 15 degrees buccal, having an abscess, a distal pit large gold filing and a buccal in-dentin carries. FIG. 12E provides an example of a display showing both a 3D representation of tooth 24 with the characteristics listed above and a pictograph chart listing the above characteristics.

### Tables:

TABLE 1
Non-limiting Examples of 3D Dentition Features of the System

| | |
|---|---|
| Teeth Lower Jaw | The system may display a fully interactive 3D model of the upper adult |
| Adult | dentition, gum levels and bone levels |
| Teeth Upper Jaw | The system may display a fully interactive 3D model of the lower adult |
| Adult | dentition, gum levels and bone levels |
| Teeth Lower Jaw | The system may display a fully interactive 3D model of the lower |
| Children | deciduous dentition, gum levels and bone levels |
| Teeth Upper Jaw | The system may display a fully interactive 3D model of the upper |
| Children | deciduous dentition, gum levels and bone levels |
| Teeth Upper Jaw | The system may display a fully interactive 3D model of the upper |
| Mixed Dentition | mixed dentition, gum levels and bone levels |
| Teeth Lower Jaw | The system may display a fully interactive 3D model of the lower |
| Mixed Dentition | mixed dentition, gum levels and bone levels |
| Complete Upper and | The system may display a 3D model of the upper and lower adult |
| Lower Jaw Adults | dentition without interactive functions |
| Complete Upper and | The system may display a 3D model of the upper and lower |
| Lower Jaw Children | deciduous dentition without interactive functions |
| Complete Upper and | The system may display a 3D model of the upper and lower mixed |
| Lower Jaw Mixed | dentition without interactive functions |
| Dentition | |

TABLE 2
Non-limiting Examples of Conditions that may have anatomically correct
Display in the Dentition Model

| | |
|---|---|
| Tooth Crown | The system may display the morphology of the crowns of teeth based |
| Anatomy | on groove and pit patterns found in the normal dentition |
| Tooth Root Anatomy | The system may display the morphology of the roots of teeth based |
| | on anatomical patterns found in the normal dentition |
| Cemento-enamel | The system may display the CEJ which delineates the boundary |
| Junction (CEJ) | between the anatomical crown and root |
| Dental Pulp | The system may display the morphology of the pulp within the crowns |
| Chamber | of teeth based normal pulp anatomy found in the normal dentition |
| Root Pulpal Canals | The system may display the morphology of the pulp canals within the |
| | roots of teeth based normal pulpal canal anatomy found in the normal |
| | dentition |
| Gingival Contours | The system may display the morphology of the gingiva found in the |
| | normal dentition |
| Alveolar Bone Levels | The system may display the morphology of the alveolar bone levels |
| | found in the normal dentition |

TABLE 3
Non-limiting Examples of Conditions that may be without
visual features to be displayed in the Dentition Model

| | |
|---|---|
| Pocket Depth | The system may record the periodontal pockets in |
| | millimeters on the 6 accepted measurement |
| | positions on every tooth |
| Temperature | The system may record the temperature of every |
| | periodontal pocket measured with the PerioBioProbe |
| | at an accuracy of +/−0.5 C. |
| Tooth Mobility | The system may have the ability to record tooth |
| | mobility based on the Miller classification of |
| | tooth mobility |
| Halitosis | The system may have the ability to record the |
| | presence of halitosis based on a 3 stage scale at |
| | 10 cm, 30 cm and 1 meter |
| Other Physiological | As the PerioBioProbe technology improves, |
| Parameters | new physiological parameters will be added |
| | to this appendix |

TABLE 4
Non-limiting Examples of Alternative presentation of the clinical information on
the Model of One Embodiment of the Present Invention.

| | |
|---|---|
| X-Ray view | The system may offer an X-Ray rendering of all the hard tissues and |
| rendering | restorations found on the 3D chart with accurate radioluscency levels |
| | for all materials |
| 2D chart view | The system may have the ability to convert the 3D chart to a standard |
| rendering | 2D charting rendering |
| Line drawing view | The system may have the ability to convert the 3D chart to a standard |
| rendering | line drawing rendering |
| Enhanced 3D view | The system may have the ability to convert the 3D chart to an |
| rendering | enhance natural looking rendering to mimic what is actually seen in |
| | the mouth |
| Bone levels without | The system may have the ability to strip away all of the soft tissues |
| gums 3D rendering | and display the bone levels around the dentition |
| Teeth only 3D | The system may have the ability to strip away all of the bone and soft |
| rendering | tissues and display only the dentition |
| Treatment History | The system may display an animation of the treatment history of the |
| Animation | patient's mouth |

TABLE 5
Non-limiting Examples of clinically relevant
information other than conditions

| | |
|---|---|
| Medical History | The system may display the patient's medical history |
| | in a questionnaire format |
| Personal medical | The system may display the patient's personal |
| information | medical information to include medications taken, |
| | allergies, alcohol and tobacco consumption, vitamins |
| | and other supplements |
| Dental history | The system may display the patient's dental history |
| | in a questionnaire format |
| Dental treatment | The system may display the patient's treatment plan |
| plan | |
| Medical history | The system may display any updates to the patient's |
| updates | medical history |

TABLE 6
Non-limiting Examples of common dental exam device outputs

| | |
|---|---|
| Intraoral Cameras | The system may interface and make available |
| | images from existing intraoral camera systems |
| Digital periapical | The system may interface and make available |
| radiographs | images from existing digital periapical radiograph |
| | systems |
| Digital panoramic | The system may interface and make available |
| radiographs | images from existing digital panoramic radiograph |
| | systems |
| Digital lateral | The system may interface and make available |
| cephalometric | images from existing digital lateral cephalometric |
| radiographs | radiograph systems |
| OCT - Optical | The system may interface and make available images |
| Coherence | from existing Optical Coherence Tomography systems |
| Tomography | |
| CAT scans | The system may interface and make available |
| | images from existing CAT scan systems |
| PerioBioProbe | The system may interface with the PerioBioProbe |
| | once it becomes commercially available |

TABLE 7
Non-limiting Examples of clinically relevant information

| | |
|---|---|
| Medical history | The system may access and display the |
| | patient's medical history |
| Dental history | The system may access and display the |
| | patient's dental history |
| Personal medical | The system may access and display the |
| information | patient's personal medical history |
| Medical history | The system may access and display the |
| updates | patient's medical history updates |
| Dental treatment | The system may access and display the |
| plan | patient's dental treatment plan |

TABLE 8
Non-limiting Examples of dental pathologies and anomalies

| | |
|---|---|
| Dental Caries | Dental caries may be indicated by a red circle on the area designated |
| | by the operator |
| Recurrent Decay | Recurrent decay may be indicated by a red circle around the existing |
| | restoration affected by decay and indicated by the operator |
| Periapical Abscesses | A periapical abscess may be indicated by a dark circular area at the |
| | tip of roots indicated by the operator. The size of the lesion (1-10 mm) |
| | may appear when indicated by the operator. |
| Gingival or | A periodontal abscess may be indicated by a dark circular area on the |
| Periodontal | lateral border of roots indicated by the operator. The size of the lesion |
| Abscesses | (1-10 mm) may appear when indicated by the operator. |
| Gingival Ulcers | Gingival ulcers may appear as white circular lesions on the gingival |
| | tissues as indicated by the operator.. The size of the lesion (1-10 mm) |
| | may appear when indicated by the operator. |
| Broken teeth | Broken teeth may appear as missing tooth segments indicated by the |
| | operator. |
| Retained roots | Retained roots may be indicated by the operator as well as the length |
| | of the retained root segment (2-10 mm) |
| Impacted Teeth | The angle and position of the impacted teeth may be indicated by the |
| | operator. Angles may be in 30 degree increments and the depth of the |
| | impaction may be indicated by the operator (1-10 mm) |
| Supernumerary | Supernumerary or extra teeth may be identified and positioned by the |
| Teeth | operator |
| Submerging Teeth | Submerging deciduous teeth may be identified by the operator and |
| | positioned from 1-10 mm. |
| Retained Deciduous | Some deciduous teeth are never lost and will be identified and |
| Teeth | positioned in the adult dentition by the operator |
| Gingival Recessions | Gingival recessions may be identified by the operator based on the |
| | position along the root of the tooth and using an incremental scale (1-15 mm) |
| Gingival Bleeding | Gingival bleeding on probing may be identified by the operator at a |
| | specific pocket area and identified as a small drop of blood |
| Gingival Pus | Gingival pus on probing may be identified by the operator at a specific |
| | pocket area and identified as a small drop of yellow pus |
| Furcation | Furcations may be identified by the operator and designated by a |
| Involvements | black circle on the furcation area of the root |
| Gingival Redness | Red gums may be identified by the operator and will appear a darker |
| | shade or red when compared to the normal gingival colour |
| Gingival Hypertrophy | Areas of gingival hypertrophy may be identified by the operator and |
| | measured according to a graduated scale (1-10 mm) |
| Gingival Hyperplasia | Areas of gingival hyperplasia may be identified by the operator and |
| | measured according to a graduated scale (1-10 mm) |
| Rotated teeth | Rotated teeth may be identified by the operator according to the |
| | direction of the rotation and may be indicated on an incremental scale |
| | at every 15 degrees |
| Tipped Teeth | Tipped teeth may be identified by the operator according to the |
| | direction of the tipping and may be indicated on an incremental scale |
| | at every 15 degrees |
| Overerupted Teeth | Overerupted teeth may be identified by the operator and may erupt |
| | incrementally on a specific scale of each mm of eruption |
| Fistulas | Fistulas may be identified by the operator according to the location of |
| | the pathology and may be indicated by a 2 mm red lesion with pus in |
| | it. |
| Tooth Abrasion | Abrasions along the CEJ may be identified by the operator and |
| | assessed a depth in 1 mm increments. They will appear as a wedge |
| | defect along the gumline. |
| Tooth erosion | Erosions along the CEJ may be identified by the operator and |
| | assessed a depth in 1 mm increments. They will appear as a circular |
| | concave defect along the gumline. |
| Occlusal wear | Occlusal wear of the teeth may be identified by the operator along the |
| | cusp tips of molars and premolars as well as the incisal edges of |
| | incisor teeth. They will be measured and reduced on the teeth by |
| | increments of 1 mm. |
| Odontomas | Odontomas may be identified and positioned by the operator. The |
| | may be designated as a white lobular mass inside the alveolar bone. |
| Tooth fusions | Fused teeth may be identified by the operator and designated as two |
| | fused crowns with separate pulp chambers and separate roots and |
| | pulpal canals. |
| Tooth geminations | Geminated or twin teeth may be identified by the operator and |
| | designated as two fused crowns with separate pulp chambers and |
| | only one root system and pulpal canal. |
| Diastemas | Diastemas or spaces between the teeth may be identified by the |
| | operator and measured in 1 mm increments. The space may be |
| | created on the chart. |

TABLE 9
Non-limiting Examples of dental restorations

| | |
|---|---|
| Amalgams | Amalgam restorations may be identified by the operator according to |
| | the tooth surface(s) covered and designated by the colour silver (mat) |
| Composite Resins | Composite resin restorations may be identified by the operator |
| | according to the tooth surface(s) covered and designated by the |
| | colour yellow (mat) |
| Porcelain Fused to | Porcelain fused to metal crowns may be identified by the operator and |
| Metal crowns | may be designated as cream coloured restoration with 45 degree line |
| | striations and a metal lingual backing |
| Full Gold Crowns | Full gold crowns may be identified by the operator and may be |
| | designated as gold coloured restorations |
| Full Metal Crowns | Full metal crowns may be identified by the operator and may be |
| | designated as white gold coloured restorations |
| Porcelain Jacket | Porcelain jacket crowns (PJC) on anterior teeth may be identified by |
| Crowns | the operator and may be designated as cream coloured restoration |
| | with 45 degree line striations |
| Full Porcelain | Full porcelain crowns on posterior teeth may be identified by the |
| Crowns | operator and may be designated as cream coloured restoration with |
| | 45 degree line striations |
| Porcelain Veneers | Porcelain veneers may be identified by the operator and may be |
| | designated as cream coloured restoration with 45 degree line |
| | striations |
| 3/4 Metal Crowns | 3/4 crowns may be identified by the operator and may be designated |
| | as gold coloured or white gold restorations. The tooth surfaces |
| | covered may be identified by the operator |
| Metal Inlays | Metal inlays may be identified by the operator and may be designated |
| | as gold coloured or white gold restorations. The tooth surfaces |
| | covered may be identified by the operator. |
| Porcelain Inlays | Porcelain inlays may be identified by the operator and may be |
| | designated as cream coloured with 45 degree striations. The tooth |
| | surfaces covered may be identified by the operator. |
| Bridge Abutments | All individual crown restorations described in Re 6.J.3 to Re 6.J.11 |
| | can be identified as bridge abutments by the operator |
| Bridge Pontics | All individual crown restorations described in Re 6.J.3 to Re 6.J.11 |
| | can be identified as bridge pontics by the operator |
| Dental Implants | Dental implants may be identified by the operator and placed in the |
| | bone where the missing tooth was. They will appear as a screw-type |
| | measuring 10 mm long by 4 mm wide post. The restoration over the |
| | implant may be identified by the operator as described in Re 6.J.3 to |
| | Re 6.J.11 |
| Maryland Bridges | Maryland bridges may be identified by the operator and designated as |
| | a porcelain fused to metal pontic (Re 6.J.13) with metal wings |
| | wrapping the buccal and lingual of posterior abutment teeth and only |
| | the lingual of anterior teeth |
| Endodontic | Endodontic treatment (root canals) may be identified by the operator |
| Treatments | and designated as pink material in the pulp chamber and the canals of |
| | the tooth |
| Endodontic posts | Posts inserted into endodontically treated teeth may be identified by |
| | the operator and designated as a metal post in tooth canals. The |
| | lengths of the posts may be incremental (5 mm to 12 mm) |
| Pit and Fissure | Pit and fissure sealants may be identified by the operator and |
| Sealants | designated as a blue colour covering the grooves of the teeth |

TABLE 10
Non-limiting Examples of non-clinical relevant information

| | |
|---|---|
| Patient Photo | A frontal view photo of the patient's face may |
| | appear on the charting interface |
| Patient Contact | The patient's contact information may appear on the |
| Information | charting interface |
| Family | Family members' information who are patients |
| Information | of the dental practice |
| Financial | A current account balance may appear on the |
| Information | charting interface. All other financial information may be |
| | accessed through a 3rd party dental system. |
| Insurance | Dental Insurance carriers names may appear on |
| Information | the charting interface. Additional insurance information |
| | may be accessed through a 3rd party dental system. |
| Scheduler | The operator's daily schedule may appear as a ribbon |
| | on the right side of the charting interface. Additional |
| | scheduling information may be accessed through a |
| | 3rd party system. |

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1. A method of recording dental information for a patient, said method comprising the steps of:
(a) providing a model, wherein said model comprises independently modifiable representations of oral structures including teeth, soft and/or bony structures, wherein each of said oral structures comprises a base structure and one or more attributes which optionally comprise one or more properties, wherein the one or more attributes may be independently activated and the one or more properties of said attributes may be independently activated to form a representation of said patient's oral/dental cavity;
(b) receiving input from a user, the input comprising one or more characteristic(s) of said patient's oral cavity;
(c) independently modifying one or more representations of oral structures of said model based on said received user input and thereby generating a dental report for said patient, wherein each of said one or more base structures to be modified is modified by activating one or more of the one or more of the attributes and optionally activating one or more of the one or more of properties of any activated attributes to produce a representation of said one or more oral structures of said patient;
(d) optionally outputting the dental report, optionally on a visual display; and
(e) optionally storing the dental report in a patient database file associated with the patient.

2. A method of updating a dental record comprising the steps of:
(a) retrieving a dental report from a patient database file located in a first database, the patient database file associated with a patient, wherein the dental report includes a model comprises independently modifiable representations of oral structures including teeth, soft and/or bony structures, wherein each of said representations of oral structures comprises a base structure and one or more attributes which may be independently activated/deactivated, wherein each of the one or more attributes optionally comprises one or more properties which may be independently activated/deactivated; wherein one or more of said representations of oral structures optionally has previously been modified in light of one or more historical characteristic(s) of said patient's oral cavity;
(b) receiving input from a user, the input comprising one or more characteristic(s) of said patient's oral cavity;
(c) comparing said one or more characteristic(s) with said one or more historical characteristic(s) of said dental model with said input and if said one or more characteristic(s) differs from said one or more historical characteristic(s) independently modifying one or more representations of oral structures of said model based on said received user input by activating/deactivating one or more of the one or more of the attributes and/or activating/deactivating one or more of the one or more of properties of said one or more attributes to produce a current representation of said one or more oral structures of said patient and thereby updating said dental report for said patient;
(d) optionally outputting the dental report, optionally on a visual display; and
(e) optionally storing the dental report in a patient database file associated with the patient.

3. The method of claim 1, wherein at least one of the one or more attributes comprises one or more of the following dental pathologies and anomalies:
Dental Caries, Recurrent Decay, Periapical Abscesses, Gingival or Periodontal Abscesses, Gingival Ulcers, Broken teeth, Retained roots, Impacted Teeth, Supernumerary Teeth, Submerging Teeth, Retained Deciduous Teeth, Gingival Recessions, Gingival Bleeding, Gingival Pus, Furcation Involvements, Gingival Redness, Gingival Hypertrophy, Gingival Hyperplasia, Rotated teeth, Tipped Teeth, Overerupted Teeth, Fistulas, Tooth Abrasion, Tooth erosion, Occlusal wear, Odontomas, Tooth fusions, Tooth germinations and Diastemas.

4. The method of claim 1, wherein at least one of the one or more attributes comprises one or more of the following dental restorations: Amalgams, Composite Resins, Porcelain Fused to Metal crowns, Full Gold Crowns, Full Metal Crowns, Porcelain Jacket Crowns, Full Porcelain Crowns, Porcelain Veneers, ¾ Metal Crowns, Metal Inlays, Porcelain Inlays, Bridge Abutments, Bridge Pontics, Dental Implants, Maryland Bridges, Endodontic Treatments, Endodontic posts and Pit and Fissure Sealants.

5. The method of claim 1, wherein at least one of the one or more attributes indicates the absence of a tooth.

6. A method of recording and/or displaying dental information for a patient, said method comprising the steps of:
providing one or more modifiable representations oral structures, wherein each of said one or more representations oral structures comprises a base structure and one or more attributes which optionally comprise one or more properties, wherein the one or more attributes may be independently activated/deactivated and the one or more properties of said attributes may be independently activated/deactivated to form a representation of said patient's oral/dental cavity; and
activating/deactivating one or more of the one or more of the attributes and optionally activating/deactivating one or more of the one or more of properties of any activated attributes to produce a representation of said dental information.

7. The method of claim 1, wherein said attributes are displayed as visual representations and/or textual representations.

8. The method of claim 1, wherein said properties are displayed as textual representations.

9. A method of displaying dental information for a patient, said method comprising the steps of:
displaying a graphical presentation of one or more oral structures, wherein each of said one or more oral structures comprises a base structure and one or more attributes which optionally comprise one or more properties, wherein the one or more attributes may be independently activated/deactivated and the one or more properties of said attributes may be independently activated/deactivated to form a representation of said patient's oral/dental cavity.

10. The method of claim 9, wherein said base structure comprises a base graphical representation, and each of said attributes comprises a graphical overlay.

11. The method of claim 1, wherein said model is three dimensional.

12. The method of claim 1, wherein said input is voice or manual.

13. A computer program product comprising a computer readable memory storing computer executable instructions thereon that when executed by a computer to perform the method of claim 1.

14. A system for electronic recording dental information for a patient, said system comprising a processor and a computer readable storage media comprising executable instructions thereon that when executed by said processor to perform the method of claim 1 12.
